(19) United States
(12) Patent Application Publication
(10) Pub. No.: US 2011/0257689 A1
(43) Pub. Date: Oct. 20, 2011

(54) BONE SCREW

(76) Inventors: Meinrad Fiechter, Munsingen (CH); Friedrich Magerl, St. Gallen (CH); Ronald Wieling, Eichberg (CH)

(21) Appl. No.: 13/141,029

(22) PCT Filed: Dec. 8, 2009
(86) PCT No.: PCT/EP2009/008743

§ 371 Date: Jun. 20, 2011

(30) Foreign Application Priority Data
Dec. 19, 2008 (EP) 08405311.5

(51) Int. Cl. A61B 17/86

(52) U.S. Cl. 606/301

(57) ABSTRACT

The invention relates to a bone screw having a screw shaft (15) comprising a point (13) and concentric to a longitudinal axis (27) of the screw, and that can be anchored in a bone or part of a bone. A screw head (14) is attached to the screw shaft (15). The screw shaft (15) is substantially made of a plastic transparent to X-rays. A part (5, 7, 9, 24) of the screw shaft (15) is made of a different material than that of a core (3, 30) substantially forming the screw shaft (15). The at least one load-bearing area (5, 7, 9, 24) is preferably made of metal or plastic, such as PEEK.

The present invention relates to a bone screw having a screw shank that has a tip and is concentric to a longitudinal axis of the screw and that is to be anchored in a bone or part of a bone, and having a screw head that is secured on the screw shank and has engagement means.

Bone screws of this kind have long been known. They are used, for example, for bone fixation by osteosynthesis. Such screws are used in particular to anchor a plate or a connecting rod to a spinal column. The screws are subject to substantial loads, for example by a clamped connection between the screw and a connecting rod or to a plate. A bone screw of this kind has been disclosed in U.S. Pat. No. 5,466,237 for example. This screw is used to secure a connecting rod to a spinal column. The connecting rod is firmly clamped between the screw head and a nut.

EP 0 507 162 A discloses a bone screw used to secure a bone plate to a spinal column. The bone screw is held in the bone plate by means of a sleeve.

EP 1 191 891 A discloses a bone screw having an axially two-part screw head. This is intended to make it easier to insert a screwdriver into the screw head.

Said bone screws are made of metal, in particular titanium. They have a high degree of strength, such that they can be subjected to high loads when being screwed in and connected to an implant. A disadvantage, however, is the lack of transparency to X-rays.

The object of the invention is to make available a bone screw of the type mentioned that has great transparency to X-rays, at least in some areas, but is still functionally reliable.

In a bone screw of the type in question, the object is achieved by the fact that the screw shank is made substantially of a plastic transparent to X-rays, and that at least one load-bearing area has a part, wherein this part is made of another material than that of a core substantially forming the screw shank. The X-ray transparency of the bone screw according to the invention is achieved by producing the screw shank from a plastic that is transparent to X-rays, for example PEEK or fiber-reinforced PEEK. Fiber-reinforced PEEK and PEEK are transparent to X-rays and relatively strong. However, fiber-reinforced PEEK is at the same time relatively brittle. A screw shank made of fiber-reinforced PEEK is nevertheless possible despite this brittleness, since at least one load-bearing area has a part made of another material. This material is metal or PEEK. This part is preferably arranged on the screw head and has said engagement means. If said part is made of metal, the bone screw lacks transparency to X-rays only in the area of the screw head. Such a screw can, like a conventional screw made of titanium, be screwed in by means of a screwdriver on the screw head and also clamped. A load-bearing area is an area that is subjected particularly to loads, in particular the screw head and/or the screw tip.

According to a development of the invention, said part is arranged on the tip of the screw shank. This part is provided in particular with a thread and permits good drilling and thread-cutting properties. The drill tip can additionally be self-tapping. Nevertheless, the bone screw according to the invention is substantially transparent to X-rays in the area of the screw shank.

According to a development of the invention, said part is sleeve-shaped. A sleeve-shaped part of this kind can be connected to the screw shank by a press fit or by adhesive bonding, for example. According to a development of the invention, the sleeve is provided in order to improve the shear strength. This sleeve is preferably arranged in a central area of the screw shank between the screw head and the tip.

The part is preferably arranged on the screw head.

However, an embodiment is also conceivable in which a first part is arranged on the screw head and a second part on the tip of the screw shank. This provides, on the one hand, very good drilling and thread-cutting properties and, on the other hand, a high load-bearing capacity of the bone screw at the screw head. In this case too, the screw shank is substantially transparent to X-rays.

Illustrative embodiments of the invention are explained in more detail below with reference to the drawing, in which:

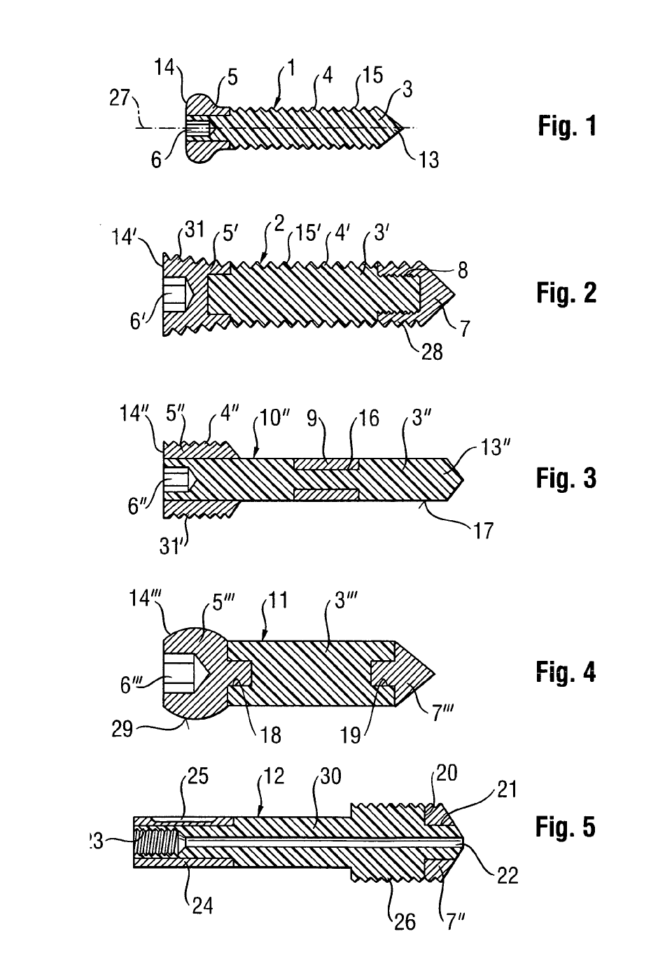

FIGS. 1-5 in each case show schematic longitudinal sections through screws according to the invention.

FIG. 1 shows a screw 1 that has a core 3 forming the screw shank. The latter has, substantially along its entire length, a thread 4 configured as an external thread. The shank 15 is coaxial with respect to a longitudinal axis 27 of the screw. The core 3 is made of a plastic transparent to X-rays, for example fiber-reinforced PEEK and in particular carbon-fiber-reinforced PEEK, or PEEK. The fibers can be long fibers or also short fibers. As can be seen, the shank 15 is concentric with respect to a longitudinal axis 27 of the screw. However, other biocompatible plastics transparent to X-rays, for example polycarbonate, polyethylene, PEK or PEEKEK, are also conceivable.

A head 14, which has a sleeve-shaped part 5, is arranged at the opposite end of the shank 15 from the tip 13. This part 5 is connected fixedly to the core 3, for example by means of a press fit or by adhesive bonding. This part 5 is made of a metal, for example of titanium or steel. If the core 3 is made of carbon-fiber-reinforced PEEK, the part 5 can be made of PEEK. Reinforcing fibers are in this case provided only in the core 3. The part 5 is the load-bearing area, for example, in the case of clamping to a plate or a connecting rod. For screwing the screw 1 into a bone or a bone part, the head 14 has an inner engagement socket 6, for example in the form of a polygonal depression. However, engagement can also be made in principle on the outside of the part 5.

FIG. 2 shows a screw 2 designed as a self-tapping screw. It likewise has a core 3′ made of fiber-reinforced PEEK or of PEEK. The core 3′ has a thread 4′, which is likewise an external thread and is used to anchor the screw 2 in a bone or a bone part. The tip of the shank 15′ is here formed by a tip 7, which is made of another material than the core 3′. The tip 7 can have a thread 28, which can be an external thread designed for self-tapping. The thread 28 supplements the thread 4′ in the area of the tip. The tip 7 is preferably made of a comparatively hard material, for example titanium or steel. The tip 7 is likewise secured to the core 3′, for example by a press fit, by adhesive bonding or by being screwed on. However, other types of connection are also possible.

The screw head 14′ is here formed by a sleeve 5′, which is likewise made of another material than the core 3′. The sleeve 5′ is fixedly connected to the core 3′. It is made, for example, of metal, in particular titanium or, in the case of a core 3′ made of fiber-reinforced PEEK, it is made of PEEK. The sleeve 5′ is provided with an inner engagement socket 6′. The screw head 14′ has a conical thread 31, which is suitable for securing in a corresponding opening of a plate (not shown here).

FIG. 3 shows a screw 10 suitable as a translaminar pin. It has a core 3″ of fiber-reinforced PEEK or PEEK. Approximately at the center between a tip 13″ and a head 14″, a circumferential recess 16 or groove is formed in the core 3″, into which circumferential recess 16 is inserted a sleeve 9. The latter is fixed at least axially and is made of another material than the core 3″. The sleeve 9 serves to increase the shear strength in the central area of the core 3″. This sleeve 9, however, can also be arranged at another part of the core 3″, for example closer to the tip 13′ or closer to the head 14″. The sleeve 9 is preferably made of a suitable metal, for example of steel or titanium. This sleeve 9 is flush with respect to a circumferential surface 17. The head 14″ has a sleeve 5″ with an external thread 4″. As can be seen, this sleeve 5″ is preferably cylindrical and is fixedly connected to the core 3″, for example adhesively bonded thereto. The head 14″ likewise has an inner engagement socket 6″.

FIG. 4 shows a screw 11 having a core 3′″ likewise made of PEEK or fiber-reinforced PEEK. At a front end, the core 3′″ has a recess 19 into which a tip 7′″ is inserted, said tip 7′″ being made of a material substantially harder than that of the core 3′″.

The core 3′″ has a further recess 18, into which a sleeve 5′″ is inserted and fixedly connected to the core 3′″. The sleeve 5′″ forms a head 14′″, which has a spherical outer surface 29 and an inner engagement socket 6′″. This screw 11 is preferably designed as a self-tapping screw. Between the tip 7′″ and the head 14′″, it is transparent to X-rays. The head 14′″ is suitable, for example, for polyaxial mounting in a peticular system. For example, the head 14′″ can be mounted and firmly clamped in a corresponding recess in a plate. The sleeve 5′″ is made, for example, of titanium or a suitable steel.

FIG. 5 discloses a femoral neck screw 12 which, as can be seen, is stepped in the longitudinal direction and, at a front end, has a greater external diameter with a cylindrical thread 26. It has a core 30, which has a passage 22 extending in the axial direction. This passage 22 is open at both ends of the core 30. At the front pointed end, the core 30 has a circumferential recess 20, into which a sleeve-shaped tip 7″ is inserted. This tip 7″ is fixedly connected to the core 30, for example by adhesive bonding. However, a connection 21 is also conceivable which is a press-fit connection or screwed connection. A sleeve 24 is formed at the end opposite the tip 7″ and is likewise fixedly connected to the core 30. This sleeve 24 has a depression 25, designed for example as a groove which extends in the axial direction and at which the screw 12 can be secured against rotation. The tip 7″ and the sleeve 24 are likewise made of another material than the core 30. The materials already mentioned above are provided. In particular, the tip 7″ and the sleeve 24 are made of a material that is substantially harder than the material of the core 30. Each of the heads 14 to 14′″ shown in FIGS. 1 to 5 can in principle be connected to each of the shanks and tips shown. Moreover, the passage 22 shown in FIG. 5 is also conceivable in the screws shown in FIGS. 1 to 4. Of course, the screws can also have markers known per se, for example of tantalum fibers, barium sulfate.

Coatings are also conceivable, for example of titanium or hydroxyapatite.

### LIST OF REFERENCE SIGNS

1 screw
2 screw
3 core
4 thread
5 sleeve
6 inner engagement socket
7 tip
8 connection (thread)
9 sleeve
10 screw
11 screw
12 screw
13 tip
14 head
15 shank
16 recess
17 circumference
18 recess
19 recess
20 recess
21 connection
22 passage
23 inner engagement socket (thread)
24 sleeve
25 depression
26 thread
27 longitudinal axis of screw
28 thread
29 outer face
30 core
31 thread

1-8. (canceled)

9. A bone screw having a longitudinal axis and comprising:
a screw shank that has a tip, is concentric to the longitudinal axis of the screw and is to be anchored in a bone or part of a bone, said screw shank being formed from a core made of a first material; and
a screw head that is secured on said screw shank and that comprises engagement means, wherein said screw shank is made substantially of a plastic material that is transparent to X-rays, and said screw has at least one part defining a load-bearing area and made of a second material that is different from the first material.

10. The bone screw as claimed in claim 9, wherein said at least one part defining a load-bearing area is made of metal or plastic

11. The bone screw as claimed in claim 10, wherein said at least one part is made of PEEK.

12. The bone screw as claimed in claim 9, wherein said at least one part is arranged on said screw head.

13. The bone screw as claimed in claim 9, wherein said at least one part forms said tip of said screw shank.

14. The bone screw as claimed in claim 9, wherein said at least one part is sleeve-shaped.

15. The bone screw as claimed in claim 9, wherein said at least one part is made of titanium or steel.

16. The bone screw as claimed in claim 9, wherein said at least one part is disposed between said screw head and said tip and is provided to increase the shear strength of said screw.

17. The bone screw as claimed in claim 9, wherein said screw is constructed as one of: a stand-alone screw; a pedicle screw; a femoral neck screw; a translaminar pin; or as a screw for securing a plate.
